(19) United States
(12) Patent Application Publication
(10) Pub. No.: US 2010/0028484 A1
(43) Pub. Date: Feb. 4, 2010

(54) METHODS FOR PRODUCING LIPIDS FROM ETHANOL PRODUCTION CO-PRODUCTS BY INTRODUCING LIPID PRODUCING MICROORGANISMS

(75) Inventors: Kevin Elliot Kriesler, Mt. Arlington, NJ (US); David James Winsness, Alpharetta, GA (US)

Correspondence Address: CANTOR COLBURN, LLP, 20 Church Street, 22nd Floor, Hartford, CT, 06103, US

(73) Assignee: GS Cleantech Corporation, New York, NY (US)

(21) Appl. No.: 12/512,708

(22) Filed: Jul. 30, 2009

Related U.S. Application Data
(60) Provisional application No. 61/084,705, filed on Jul. 30, 2008.

(51) Int. Cl. A23D 7/00; C12P 7/64; C12P 7/06

(52) U.S. Cl. 426/7; 435/134; 435/161

(57) ABSTRACT

Methods for producing a lipid rich product from a feedstock utilized in wet and dry milling processes for producing ethanol, the method include mixing a culture of lipid producing microorganisms with the feedstock, wherein the feedstock includes co-products of ethanol production and/or biomass; producing lipids within the lipid producing microorganisms; lysing the microorganisms; and isolating the lipid rich product.

### CROSS REFERENCE TO RELATED APPLICATIONS

The present application relates to and claims priority to U.S. Provisional Application No. 61/084,705 filed on Jul. 30, 2008, incorporated herein by reference in its entirety.

### BACKGROUND

The present disclosure generally relates to the production and recovery of lipids from traditional wet milling, dry milling and fractionated dry milling ethanol production facilities through the use of lipid producing microorganisms.

Over the past thirty years, significant attention has been given to the production of ethyl alcohol, i.e., ethanol, for use as an alternative fuel. Ethanol not only burns cleaner than fossil fuels, but also can be produced using grains such as corn, which is a a renewable resource in abundant supply. Ethanol can be produced from various grains such as corn by either a wet milling process or a dry mill process.

In the wet milling process, the corn kernels are separated into different components such as germ, starch, protein, corn oil, gluten meal, gluten feed, distillers solubles and fiber, resulting in several co-products that can be further processed to yield valuable materials. For example, separated germ can be further processed for lipid recovery; starch can be saccharified and fermented for ethanol production; and protein and fiber can be used as animal feed material.

In a traditional dry mill process, as is generally shown in FIG. 1, the corn is not fractionated and generally only two co-products are produced in addition to ethanol, which are distillers grains and carbon dioxide (CO₂). In the traditional dry mill process, the entire ground corn kernel is processed through fermentation and distillation to produce ethanol, whole stillage, and CO₂. The whole stillage contains water and distillers grains, which generally includes a portion of the starch that was not fermented, and the remaining non-fermentable portions of the kernel of corn such as protein, fiber, cellulose, lignocellulose and hemicellulose, corn lipids and ash. Water is typically removed from the whole stillage to form dried distillers grains (“DDG”). At present, an estimated one hundred and thirty dry milling plants are producing about 10 billion gallons of ethanol per year, an amount which is expected to grow to 15 billion gallons of ethanol per year by 2015.

Technology exists today that effectively recovers corn lipids (i.e., fats or oil) from the whole stillage, thin stillage, concentrated thin stillage, wet distillers grains and dry distillers grains produced by dry mill ethanol facilities. These processes generally include solvent extraction, membrane filtration, centrifugation, and the like. FIG. 2 schematically illustrates an exemplary solvent extraction process scheme. The whole stillage is dried (moisture content removed) and the oil is then extracted from the concentrated stillage. The extraction process may include, but is not limited to solvent extraction, press extraction and/or supercritical extraction. The extracted lipids provide a significant financial resource to the ethanol facility while helping to fill a starved pipeline of feedstock to biodiesel and renewable diesel companies. The extraction of lipids as described above increases the amount of fuel derived from an equivalent mass of corn while decreasing the energy needs to produce fuel and further advances the industry toward a goal of producing carbon-neutral fuels. However, current processes are generally not efficient and/or use chemical, e.g., solvents that require careful control and selection.

FIG. 3 schematically illustrates a dry mill ethanol production process including a lipid extraction step prior to drying. The lipid extraction methods generally include but are not limited to membrane separation, centrifugation, heat conditioning prior to centrifugation, washing, and any combination thereof

While most of the current ethanol production facilities in use are dry mill facilities, there has been a slowly developing trend to build “fractionation-based” dry milling ethanol production facilities. These fractionated facilities attempt to separate as much of the non-fermentable portions of the grain as practical prior to the fermentation step. For example, corn kernels are comprised of three primary components: endosperm, germ, and bran. The endosperm contains the majority of the starch within the kernel of corn, typically about 85%, whereas the germ and the bran contain high concentrations of non-fermentables, e.g., fiber, protein, and corn oil. Wet and dry fractionation technologies exist today that can be integrated into the dry milling process to separate the endosperm, germ, and bran with minimal losses. The separated endosperm can then be conveyed to the fermentation process, and the germ and bran can then be sold directly to other markets and/or further processed. With less non-fermentable mass entering the fermentation vessels, greater volumes of ethanol can be produced per volume of fermentation capacity. In addition, separating non-fermentables prior to fermentation allows for a reduced mass of whole stillage exiting distillation and advantageously reduces energy loads on the whole stillage dehydration equipment utilized for drying. The downside of the current technology is that the separation equipment and processes used need improvements to make the processes commercially viable. For example, some of the starch exits with the non-fermentable components, thereby increasing the mass of corn required per volume of ethanol produced. This may be satisfactory as long as the non-fermentable co-products retain favorable value and ethanol production capacity increases relative to the reduced non-fermentables in the process. However, the objective of the development of fractionated dry milling ethanol facilities is to increase co-product value, decrease energy consumption, and to create additional valuable co-products such as corn oil. Fractionation upgrades have not occurred as frequently as back-end lipid extraction due to the substantial capital requirements and are yet to provide proven energy reductions. A substantial amount of research and development continues to occur and the technology may become more widely accepted as the methods are proven and accepted.

In both the traditional dry milling production facility and the fractionated dry milling ethanol production facility, the whole stillage is typically dehydrated by separating the heavy phase from the lighter phase using a centrifuge, a screen, a rotary screen, or a press of some sort. The heavier phase is commonly referred to by those in the art as wet distillers grains and the lighter phase is commonly referred to as thin stillage. The thin stillage can then be concentrated efficiently using multi-effect evaporation to produce a product generally referred to as condensed distillers solubles and/or thin stillage concentrate.

It was previously believed that the maximum mass of lipids that can be recovered from these prior milling processes was no greater than the lipids contained in the grain itself. Accordingly, it would be a significant commercial advantage and advance to increase the yield of lipids obtained over the maximum theoretical yield.

### BRIEF SUMMARY

Disclosed herein are methods for producing a lipid rich product from a feedstock utilized in wet and dry milling processes for producing ethanol.

In one embodiment, the method comprises mixing a culture of lipid producing microorganisms with the feedstock, wherein the feedstock comprises whole grains and/or co-products of ethanol production and/or biomass; producing a lipid rich product within the lipid producing microorganisms from the whole grains and/or co-products of ethanol production and/or biomass; and isolating the lipid rich product.

The above described and other features are exemplified by the following figures and detailed description.

### BRIEF DESCRIPTION OF THE DRAWINGS

Referring now to the figures wherein like elements are numbered alike:

PRIOR ART FIG. 1 schematically illustrates a typical dry mill ethanol production process commonly employed in grain-to-ethanol producing facilities.

PRIOR ART FIG. 2 schematically illustrates a dry mill ethanol production process with the addition of a lipid solvent extraction method installed post drying of the DDGS.

PRIOR ART FIG. 3 schematically illustrates a dry mill ethanol production process including a lipid extraction step prior to drying. The lipid extraction methods include but are not limited to membrane separation, centrifugation, heat conditioning prior to centrifugation, washing and any combination thereof.

FIG. 4 schematically illustrates a milling process in accordance with the present invention that includes the addition of a second fermentation reactor for lipid production. lipid producing microorganisms are introduced to the whole stillage, thin stillage or concentrated thin stillage of traditional dry mill or fractionated ethanol production facilities and oil recovery occurring post drying. The second fermentation may be more efficient through the use of a conditioning step prior to fermentation.

FIG. 5 schematically illustrates addition of a second fermentation reactor where lipid producing microorganisms are introduced to the whole stillage, thin stillage or concentrated thin stillage of traditional ethanol production facilities and oil recovery occurring prior to drying. The fermentation may be more efficient through the use of a conditioning step prior to fermentation as described above.

FIG. 6 schematically illustrates preferred processing technique of the inventors and includes the immediate extraction of oil from the stillage post ethanol distillation and then the addition of a second fermentation reactor where lipid producing microorganisms are introduced. After the second fermentation reactor an additional oil extraction process is used. After oil extraction, the remaining product may or may not be dried further.

FIG. 7 schematically illustrates a front end fractionated process. Whole grains, such as corn, can be separated into endosperm, germ and bran. The endosperm can be processed in lieu of corn in FIGS. 1-6 and the remaining germ and bran can be sold or further processed. The bran, for example, can be conditioned as described in paragraph 9 prior to the introduction of lipid producing microorganisms and then oil can be recovered from the microorganisms. The germ can be processed for oil recovery independently or in conjunction with the bran or microorganisms.

FIGS. 8A and 8B schematically illustrate replacement of traditional ethanol fermentation reactors with lipid producing microorganisms for the production of lipids in lieu of ethanol and use of the ethanol fermentation reactors in conjunction with the processing of whole grain by lipid producing microorganisms for the production of lipids.

FIG. 9 schematically illustrates replacement of traditional ethanol fermentation reactors with lipid producing microorganisms for the production of lipids in lieu of ethanol. In this figure, oil extraction occurs after to drying.

FIG. 10 schematically illustrates contribution of additional grains and/or other forms of biomass in a process designed to enhance lipid production and recovery in addition to increasing the protein, fiber and/or nutrient content of distillers grain natively produced by the ethanol facility.

### DETAILED DESCRIPTION

Disclosed herein are processes for converting at least a portion of the non-ethanol co-products of dry or wet mill ethanol production processes, whole grains, and/or biomass into lipids by processing the non-ethanol co-products, whole grains, and/or biomass with lipid producing microorganisms, extracting lipids from these microorganisms, and optionally refining the extracted lipids into renewable fuels. The term “lipid” generally refers to a class of hydrocarbons that are soluble in non-polar solvents and are relatively or completely insoluble in water. Lipid molecules have these properties because they consist largely of long hydrocarbon tails which are hydrophobic in nature. Examples of lipids include fatty acids (saturated and unsaturated); glycerides or glycerolipids (such as monoglycerides, diglycerides, triglycerides or neutral fats, and phosphoglycerides or glycerophospholipids); nonglycerides (sphingolipids, sterol lipids including cholesterol and steroid hormones, prenol lipids including terpenoids, fatty alcohols, waxes, and and polyketides); and complex lipid derivatives (sugar-linked lipids, or glycolipids, and protein-linked lipids). Fats are a subgroup of lipids commonly referred to as triacylglycerides. The term “biomass” generally refers to plant material, vegetation, and/or agricultural waste that can be used as a fuel or energy source.

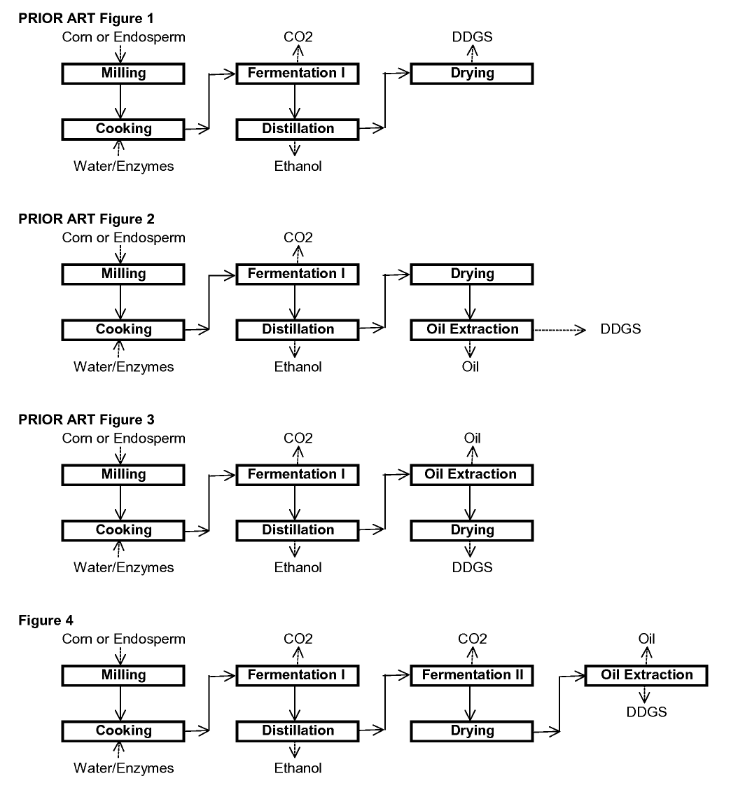

In one embodiment as shown in FIG. 4, the process includes the mixing the lipid producing microorganisms such as Rhodotorula glutinis in a second fermentation tank that includes a suitable medium such as water with the non-ethanol products obtained after drying. The lipid producing microorganisms consume and convert the non-ethanol co-products of to lipids. In this manner, yields greater than the theoretical mass of lipids of the grains itself can be obtained by conversion of the non-ethanol co-product from the respective milling process. Advantageously, and as will be discussed herein, the introduction of the lipid microorganisms can utilize the existing infrastructure utilized in ethanol production facilities so as to make the process commercially viable. No significant equipment and/or capital costs are needed to integrate the introduction of the lipid producing microorganisms in the dry, wet or fractionated milling processes. The production of additional lipids from within the existing co-products and recovery of these lipids further increases the output on a per bushel of feedstock basis while decreasing the energy consumption to produce a given volume of fuel. Moreover, the medium of the dried stillage typically includes water and provides a hydration source for the lipid producing microorganisms. In one embodiment, mixing the culture of the lipid producing microorganism is at a temperature between 5° C. and 80° C., and in other embodiments, between 15° C. and 50° C.

Isolation of the lipids can be effected by lysing the lipid producing microgranisms after a suitable amount of time to produce a lysate that is lipid rich. Lysing can be achieved by conventional means including, without limitation, heat-induced lysis, adding a base, adding an acid, using enzymes such as proteases and polysaccharide degradation enzymes such as amylases, using ultrasound, mechanical lysis, using osmotic shock, infection with a lytic virus, and/or expression of one or more lytic genes. In other embodiments, isolating the lipid rich product comprises solvent extraction.

The particular lipid producing microorganisms are not intended to be limited in this and any embodiments disclosed herein. The lipid producing microorganisms can include a single type of microorganisms or a mixture of microorganisms. Any species of microorganism that produces suitable lipid or hydrocarbon can be used, although microorganisms that naturally produce high levels of suitable lipid or hydrocarbon are preferred. Production of hydrocarbons by microorganisms is reviewed by Metzger et al., Appl Microbiol Biotechnol (2005) 66: 486-496 and A Look Back at the U.S. Department of Energy's Aquatic Species Program: Biodiesel from Algae, NREL/TP-580-24190, John Sheehan, Terri Dunahay, John Benemann and Paul Roessler (1998). The lipid producing microorganism can be algae, yeast, fungi, bacteria, and various combinations thereof. It should be apparent that the use of the lipid producing microorganisms can produce lipids that are structurally different from corn oil, thereby providing an opportunity to produce higher grades of oil, e.g., oils that have higher nutrient values.

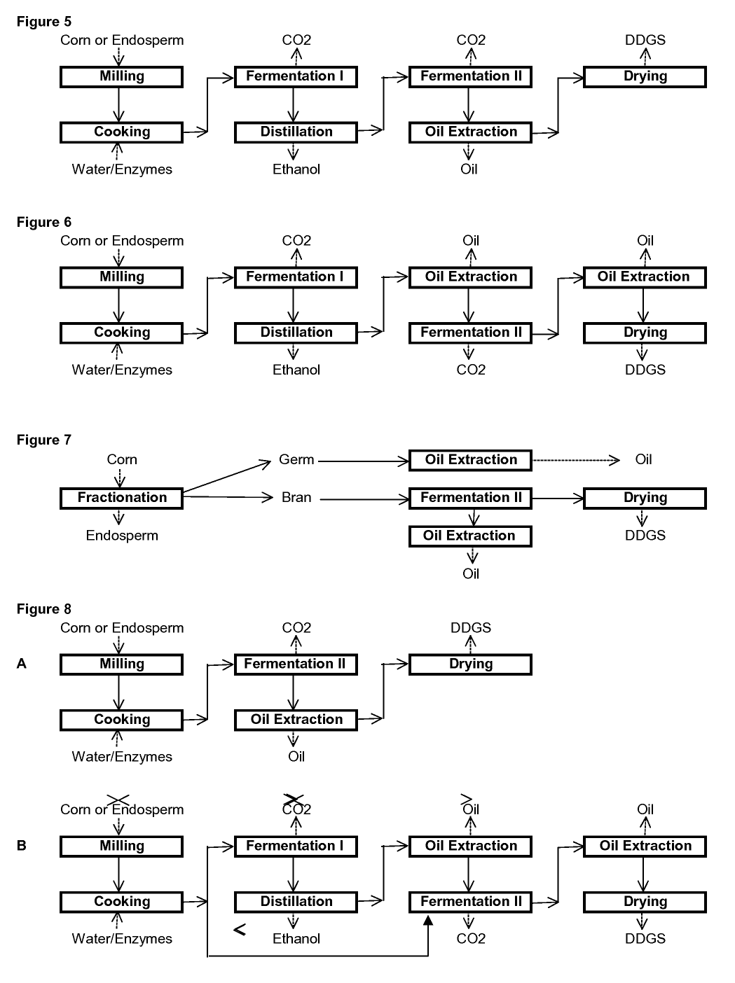

In another embodiment as shown in FIG. 5, the process includes the addition of lipid producing microorganisms after ethanol recovery/distillation to the remaining whole stillage, thin stillage, and/or concentrated thin stillage to allow the lipid producing microorganisms to consume additional sugars and biomass; and then applying lipid recovery methods to the mixture. In other words, the lipid producing microorganisms are added after fermentation (I) of the starch to ethanol and prior to drying. The oil can then be extracted from the lipid producing microorganisms in the manner previously described The illustrated process can utilize existing infrastructure where whole stillage, thin stillage and/or concentrated thin stillage tanks are used or expanded to serve as fermentation and/or digestion vessels for the lipid producing microorganisms. Additionally, as the fermentation and/or digestion process occurs in a hydrated state, there may not be a need to consume additional volumes of water as the thin and whole stillage mixtures contain sufficient levels of moisture. Furthermore, the lipid extraction process used to recover lipids from the microorganisms can be very similar to the existing methods used to recover lipids from the co-products of ethanol production prior to the introduction of lipid producing microorganisms, thereby further reducing the potential needs of process equipment and engineering design as substantial amounts of this infrastructure exists, is scaleable, and is readily available.

In another embodiment as shown in FIG. 6, the process includes extracting a portion of the existing lipids from within the whole stillage, thin stillage and/or concentrated thin stillage obtained after starch fermentation, i.e., prior to the addition of lipid producing microorganisms to the remaining defatted material. The process may include a conditioning step as described above prior to the introduction of lipid producing microorganisms. The conditioning step generally includes subjecting the feedstock material to a process to free or create sugars and/or other sources of carbon, and/or to increase nutrient availability from components such as cellulose, lignocellulose, hemicelluloses and unfermented starch to allow for increased production and growth of lipid producing microorganisms. Exemplary conditioning processes include, but are not limited to, steam explosion, autohydrolysis, ammonia fiber expansion, acid hydrolysis, ultrasonication, irradiation (for example, with microwave bombardment, or directed electromagnetic stimulation), hydrodynamic shock, cavitation, enzymatic conditioning, or combinations thereof.

This conditioning step can potentially allow the production of two chemically different lipid streams, wherein the lipids are structurally different. Advantageously, the process can minimize yield losses as introduction of the lipid producing microorganisms prior to extraction of the already available lipids could allow the microorganism to consume existing lipids present in the feedstock, which can result in new lipid yield loss due to conversion of native lipids into new lipids and non-lipid products. Thus, a preceding lipid extraction step on the naturally available lipids before the introduction of lipid producing microorganisms may be desirable in some applications.

In another embodiment as shown in FIG. 7, the process includes fractionating the whole grain prior to ethanol fermentation. The fractionation step primarily produces germ, bran and endosperm. Any of these products can then be converted or partially converted into lipids through the introduction of a culture of the lipid producing microorganisms. Generally, the germ would be processed through a lipid extraction system prior to being introduced to lipid producing microorganisms to enhance overall efficiency. Additionally, the biomass may be conditioned prior to the introduction of the lipid producing microorganisms to allow for greater conversion efficiency. Lastly, the lipid producing microorganisms may be introduced to the endosperm as it may be advantageous to convert this product into lipid products in lieu of ethanol fermentation, i.e., in some applications it may be desirable to produce lipids as opposed to ethanol.

In another embodiment as shown in FIGS. 8A and 8B, the process includes the use of one or more lipid producing microorganisms on the whole grain, either in lieu of (FIG. 8A) or in conjunction with a conventional fermentation process (FIG. 8B) utilizing ethanol producing microorganisms, to convert as much of the whole grain into lipid as possible. The whole grain may be conditioned as previously described prior to introduction of the lipid producing microorganisms.

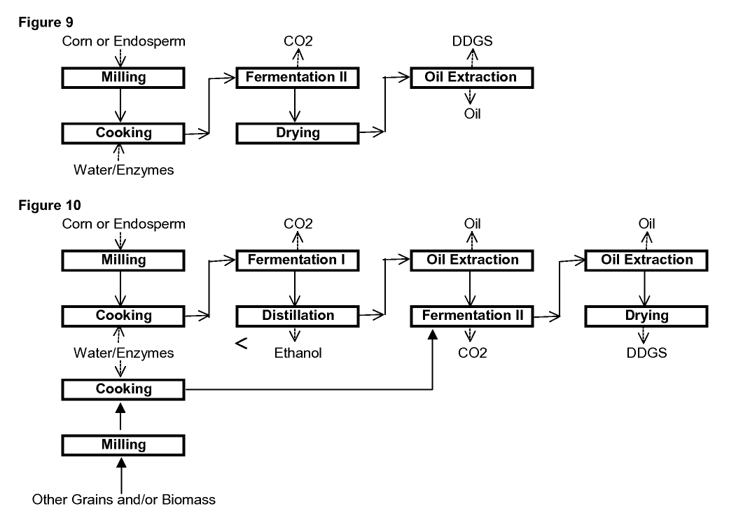

In another embodiment as shown in FIG. 9, the process includes the introduction of other grains, such as barley, wheat and the like, and/or other biomass, such as corn cobs, corn stover, grasses and the like, containing additional qualified sources of carbon (such as sugars, starch, cellulose, native lipids, protein, minerals and/or other nutrients) to the lipid producing microorganisms. This additional grain and/or biomass flow would be processed in parallel, in series, or in combination with the whole stillage, thin stillage or concentrated thin stillage flow of a contiguous conventional ethanol production process, and then be processed for lipid recovery with the host ethanol facility's lipid extraction equipment. This technique would enable increased levels of lipid production and extraction as compared to, for example, the lipid production levels made possible as described above in addition to enhancing the protein, fiber and nutrient content of the distillers grain natively produced by the ethanol facility.

In another embodiment, as shown in FIG. 10, the process includes extracting a portion of the existing lipids from within the whole stillage, thin stillage and/or or concentrated thin stillage prior to the addition of lipid producing microorganisms to the remaining material. This process may include a conditioning step prior to the introduction of lipid producing microorganisms and the process could also include the introduction of other grains, such as barley, wheat and the like, and/or other biomass containing additional qualified sources of carbon (such as sugars, starch, cellulose, native lipids, protein, minerals and/or other nutrients) to the material. This additional grain and/or biomass flow would be combined with the whole stillage, thin stillage and/or concentrated thin stillage flow after extraction of the lipids naturally available in the host ethanol plant's traditional feedstock (such as corn),and then processed for lipid recovery prior to introduction of the lipid producing microorganisms. This technique would enable increased levels of lipid production and extraction as compared to, for example, the lipid production levels made possible by the embodiment described above in addition to enhancing the protein, fiber and nutrient content of the distillers grain natively produced by the ethanol facility.

It should be apparent that the use of the lipid producing microorganisms may be targeted as a process to modify the nutrient content of the previously produced whole stillage, thin stillage, concentrated thin stillage, wet distillers grains, dried distillers grains or dried distillers grains with solubles. Conversion of a portion of the targeted feedstock by one or more lipid producing microorganisms can modify the nutritional qualities of the resulting product, including, for example, the production of lipids with specifically-tailored free fatty acid profiles, and co-product grains with specific amino acid profiles.

The use of lipid producing microorganisms may also be used as a means to reduce the energy costs of dehydrating the co-products of ethanol production. By converting a portion of the co-products of ethanol production into lipids (and carbon dioxide in the case of aerobic microorganisms), and then extracting those lipids prior to the dewatering and drying stages of conventional ethanol production, the corresponding reduction in co-product mass in need of dewatering and drying will result in a reduction of the host ethanol facility's energy consumption.

While the disclosure has been described with reference to an exemplary embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

What is claimed:

1. A method for producing a lipid rich product from a feedstock utilized in milling processes for producing ethanol, the method comprising:
mixing a culture of lipid producing microorganisms with the feedstock, wherein the feedstock comprises whole grains and/or co-products of ethanol production and/or biomass;
producing a lipid rich product within the lipid producing microorganisms from the whole grains and/or co-products of ethanol production and/or biomass; and
isolating the lipid rich product.

2. The method of claim 1, wherein the lipid producing microorganisms comprise Rhodotorula glutinis.

3. The method of claim 1, wherein the mill process is free of a fractionation step of the whole grain prior to fermentation.

4. The method of claim 3, wherein the feedstock comprises, individually or in combination, whole stillage, partially defatted whole stillage, thin stillage, partially defatted thin stillage, concentrated thin stillage, partially defatted thin stillage, wet distillers grain, partially defatted wet distillers grain, dry distillers grain, partially defatted dry distillers grain, dry distillers grain with solubles, bran, endosperm, germ, oil extracted germ, starch, gluten meal, corn oil, distillers solubles, wet corn gluten feed, corn stover, corn cobs, grasses, leaves, wood, and dry corn gluten feed.

5. The method of claim 1, wherein the milling process is a dry milling process comprising a dry or wet fractionation step prior to a fermentation step.

6. The method of claim 5, wherein the fermentation step comprises mixing the lipid producing microorganisms with a fraction to produce a lipid rich product.

7. The method of claim 1, further comprising conditioning the feedstock prior to mixing the culture of the lipid producing microorganism with the feedstock.

8. The method of claim 7, wherein the conditioning comprises steam explosion, autohydrolysis, ammonia fiber expansion, acid hydrolysis, ultrasonication, irradiation (for example, with microwave bombardment, or directed electromagnetic stimulation), hydrodynamic shock, cavitation, enzymatic conditioning, or combinations thereof.

9. The method of claim 1, wherein mixing the culture of the lipid producing microorganism is at a temperature between 5° C. and 80° C.

10. The method of claim 1, wherein isolating the lipid rich product from the lipid producing microorganisms comprises solvent extraction.

11. The method of claim 1, wherein isolating the lipid rich product from the lipid producing microorganisms comprises lysing, membrane separation, centrifugal separation, super critical extraction, or press extraction.

12. The method of claim 11, further comprising pretreating the lipid producing microorganisms subsequent to producing the lipid rich product within the lipid producing microorganisms, wherein pretreating comprises washing to free lipids contained within the solids, heating, separating a heavy phase from a light phase, and/or evaporation.

13. The method of claim 1, wherein the lipid rich products are further processed to produce fuel.

14. The method of claim 1, wherein the lipid rich products are further refined into edible oils.

15. The method of claim 1, wherein after isolating the lipid rich product and animal feed material remains.

16. The method of claim 1, wherein the lipid producing microorganism is separated from the feedstock after fermentation through centrifugation or membrane filtration.

17. The method of claim 1, wherein the whole grain is corn and/or milo.

18. The method of claim 1, wherein the lipid producing microorganisms are hydrated prior to mixing the culture of the lipid producing microorganisms with the feedstock wherein hydration comprises recycling water within a facility configured for ethanol production, and/or hydrated by the whole stillage, thin stillage, concentrated thin stillage, partially defatted whole stillage, partially defatted thin stillage or partially defatted concentrated thin stillage.

19. The method of claim 1, wherein the lipid producing microorganisms consume feedstock mass, increase lipid concentrations, and reduce mass by removal of the CO₂ generated.

20. The method of claim 19, where reducing the mass reduces drying energy requirements.

21. The method of claim 18, wherein the lipid rich product is partially recovered to further reduce mass and the drying energy requirements prior to drying.

22. The method of claim 1, wherein the lipid producing microorganisms alter the chemical properties of the feedstock.

23. The method of claim 1, further comprising adding nutrients to the culture in an amount effective to enhance productivity of the microorganisms.

24. The method of claim 1, wherein the lipid rich product is different from corn oil.
